(19) United States
(12) Patent Application Publication
(10) Pub. No.: US 2003/0083262 A1
(43) Pub. Date: May 1, 2003

(54) Methods and compositions for treating inflammatory disorders

(75) Inventors: Gerhard Hannig, Revere, MA (US); Douglas Lazarus, Watertown, MA (US)

Correspondence Address: LAHIVE & COCKFIELD, 28 STATE STREET, BOSTON, MA, 02109, US

(73) Assignee: Praecis Pharmaceuticals Inc., Waltham, MA (US)

(21) Appl. No.: 10/229,915

(22) Filed: Aug. 27, 2002

Related U.S. Application Data
(60) Provisional application No. 60/316,328, filed on Aug. 30, 2001.

(51) Int. Cl. A61K 38/10; A61K 38/08

(52) U.S. Cl. 514/14; 514/15; 514/16; 514/17

(57) ABSTRACT

The present invention provides methods and compositions for treating inflammatory disorders, e.g., asthma, lung inflammation or cancer.

### RELATED APPLICATIONS

This application claims priority to U.S. Provisional Patent Application Serial No. 60/316,328 filed Aug. 30, 2001, the entire contents of which are incorporated herein by reference.

### BACKGROUND OF THE INVENTION

Inflammation is defined as the reaction of vascularized living tissue to injury. As such, inflammation is a fundamental, stereotyped complex of cytologic and chemical reactions of affected blood vessels and adjacent tissues in response to an injury or abnormal stimulation caused by a physical, chemical or biological agent. Inflammation usually leads to the accumulation of fluid and blood cells at the site of injury, and is usually a healing process. However, inflammation sometimes causes harm, usually through a dysfunction of the normal progress of inflammation. Inflammatory diseases are those pertaining to, characterized by, causing, resulting from, or becoming affected by inflammation. Examples of inflammatory diseases or disorders include, without limitation, asthma, lung inflammation, chronic granulomatous diseases such as tuberculosis, leprosy, sarcoidosis, and silicosis, nephritis, amyloidosis, rheumatoid arthritis, ankylosing spondylitis, chronic bronchitis, scleroderma, lupus, polymyositis, appendicitis, inflammatory bowel disease, ulcers, Sjorgen's syndrome, Reiter's syndrome, psoriasis, pelvic inflammatory disease, orbital inflammatory disease, thrombotic disease, and inappropriate allergic responses to environmental stimuli such as poison ivy, pollen, insect stings and certain foods, including atopic dermatitis and contact dermatitis.

Inflammatory diseases present a worldwide problem. Studies of disease burden have re-affirmed that tuberculosis is among the top 10 causes of death in the world. Asthma affects 5% of the adult population and 10-15% of the population of children (Armetti and Nicosia (1999) Boll Chim. Farm. 138(11):599). Asthma is a chronic inflammatory disease that is associated with widespread but variable airflow obstruction.

Sepsis is yet another inflammatory disorder and is caused by the presence of various bacterial cell wall components in the blood or tissues of a subject. Sepsis is characterized by a systemic inflammatory response to bacterial products during infection. The symptoms of sepsis, such as fever, are caused at least in part by the inflammatory response of the body to the infecting agent.

Accordingly, there is still a great need for methods and compounds useful for treating inflammatory disorders.

### SUMMARY OF THE INVENTION

The present invention provides anti-inflammatory compounds, pharmaceutical compositions thereof, and methods of use thereof for treating inflammatory disorders. The present invention is based, at least in part, on the discovery that cell membrane-permeable polybasic peptides have therapeutic activity in animal models of inflammation.

Accordingly, in one aspect, the present invention provides a method of treating an inflammatory disorder, e.g., asthma, lung inflammation or cancer, in a subject. The method includes administering to the subject a therapeutically effective amount of a polybasic peptide, e.g., a cell membrane-permeable polybasic peptide, thereby treating an inflammatory disorder in a subject. Preferably, the polybasic peptide comprises from 5 to 16 amino acid residues, more preferably from 5 to 12 residues and, most preferably, from 7 to 11 residues. In one embodiment, the polybasic peptide comprises the third helix of the antennapedia homeodomain protein, or a fragment or variant thereof, or amino acid residues 48-57 of the HIV tat protein or fragment or variant thereof. In another embodiment, the polybasic peptide is derived from gelsolin. Preferably, at least 30%, 40%, 50%, 60%, 70%, or 80% of the amino acid residues in the polybasic peptide are independently selected from lysine and arginine residues. In one embodiment, the polybasic peptide includes no more than four contiguous non-basic amino acid residues and, preferably, no more than three contiguous non-basic amino acid residues.

In another embodiment, the anti-inflammatory compounds of the present invention may further include a modifying group, e.g., a C-terminal modifying group such as an —NH₂ group, an —NH(alkyl) group, an —N(alkyl)₂ group, or an alkoxy group; or an N-terminal modifying group such as an acyl group; or one or two alkyl group groups.

In one embodiment, the anti-inflammatory compounds used in the methods of the invention have the structure:

B₁—X₁—X₂—X₃—B₂—X₄—X₅—B₃,

wherein B₁, B₂ and B₃ are each, independently, a basic amino acid residue and X₁, X₂, X₃, X₄ and X₅ are each, independently, an alpha-helix promoting amino acid residue. In one embodiment, at least one of B₁, B₂ and B₃ is arginine; preferably B₁, B₂ and B₃ are each arginine. In another embodiment, at least one of X₁, X₂, X₃, X₄ and X₅ is alanine; preferably all of residues X₁-X₅ are alanine.

In another embodiment, the anti-inflammatory compounds used in the methods of the invention have the structure:

B₁—X₁—X₂—B₂—B₃—X₃—X₄—B₄,

wherein B₁, B₂, B₃ and B₄ are each, independently, a basic amino acid residue and X₁, X₂, X₃ and X₄ are each, independently, an alpha-helix promoting amino acid residue. In one embodiment, at least one of B₁, B₂, B₃ and B₄ is arginine; preferably B₁, B₂, B₃ and B₄ are each arginine. In another embodiment, at least one of X₁, X₂, X₃ and X₄ is alanine; preferably all of residues X₁-X₄ are alanine.

In a further aspect, the present invention provides anti-inflammatory compounds having a peptide sequence selected from the group consisting of: DRQIKIWFQNRRMKWKK (SEQ ID NO:1); RQIKIWFQNRRMKWKK (SEQ ID NO:2); QIKIWFQNRRMKWKK (SEQ ID NO:3); IKIWFQNRRMKWKK (SEQ ID NO:4); KIWFQNRRMKWKK (SEQ ID NO:5); IWFQNRRMKWKK (SEQ ID NO:6); WFQNRRMKWKK (SEQ ID NO:7); FQNRRMKWKK (SEQ ID NO:8); QNRRMKWKK (SEQ ID NO:9); NRRMKWKK (SEQ ID NO:10); RRMKWKK (SEQ ID NO:11); FKSGLKYKK (SEQ ID NO:12); KSGLKYKK (SEQ ID NO:13); QRLFQVKGRR (SEQ ID NO:14); RLFQVKGRR (SEQ ID NO:15); YGRKKRRQRRRP (SEQ ID NO:16); GRKKRRQRRRP (SEQ ID NO:17); RKKRRQRRRP (SEQ ID NO:18); RKKRRQRRRPGG (SEQ ID NO:19); AGRKKRRQARR (SEQ ID NO:20); YARKARRQARR (SEQ ID NO:21); YARAAARQARA (SEQ ID NO:22); YARAARRAARR (SEQ ID NO:23); YARAARRAARA (SEQ ID NO:24); YARRRRRRRRR (SEQ ID NO:25); RKKRRQRRR (SEQ ID NO:26); RKKRRQRR (SEQ ID NO:27); YGRKKRRQRRR (SEQ ID NO:28); YGRKKRRQRR (SEQ ID NO:29); GRKKRRQRRR (SEQ ID NO:30); GRKKRRQRR (SEQ ID NO:31); RRRRR (SEQ ID NO:32); RRRRRR (SEQ ID NO:33); RRRRRRR (SEQ ID NO:34); RRRRRRRR (SEQ ID NO:35); RRRRRRRRR (SEQ ID NO:36); RRRRRRRRRR (SEQ ID NO:37); RRRRRRRRRRR (SEQ ID NO:38); RRRRRRRRRRRR (SEQ ID NO:39). Methods of using the foregoing peptides for treating an inflammatory disorder are also provided by the present invention.

Pharmaceutical compositions and kits containing the anti-inflammatory compounds of the present invention are also provided by the present invention.

Other features and advantages of the invention will be apparent from the following detailed description and claims.

### DETAILED DESCRIPTION OF THE INVENTION

The present invention provides anti-inflammatory compounds, pharmaceutical compositions thereof, and methods of use thereof for treating inflammatory disorders. The present invention is based, at least in part, on the discovery that cell membrane-permeable polybasic peptides have therapeutic activity in animal models of inflammation.

In one embodiment, the invention provides a method of treating an inflammatory disorder, e.g., asthma, lung inflammation or cancer, in a subject. The method comprises administering to the subject a therapeutically effective amount of a polybasic peptide, e.g., a cell membrane-permeable polybasic peptide, thereby treating an inflammatory disorder in a subject.

As used herein, an “inflammatory disorder” is intended to include a disease or disorder characterized by, caused by, resulting from, or becoming affected by inflammation. An inflammatory disorder may be caused by or be associated with biological and pathological processes associated with, for example, NF-κB mediated processes. Examples of inflammatory diseases or disorders include, but are not limited to, acute and chronic inflammatory disorders such as asthma, psoriasis, rheumatoid arthritis, osteoarthritis, psoriatic arthritis, inflammatory bowel disease (Crohn's disease, ulcerative colitis), ankylosing spondylitis, sepsis, vasculitis, and bursitis; autoimmune diseases such as Lupus, Polymyalgia, Rheumatica, Scleroderma, Wegener's granulomatosis, temporal arteritis, cryoglobulinemia, and multiple sclerosis; transplant rejection; osteoporosis; cancer, including solid tumors (e.g., lung, CNS, colon, kidney, and pancreas); Alzheimer's disease; atherosclerosis; viral (e.g., HIV or influenza) infections; chronic viral (e.g., Epstein-Barr, cytomegalovirus, herpes simplex virus) infection; and ataxia telangiectasia.

Further examples of inflammatory diseases or disorders include those diseases with an NF-κB inflammatory component. Such diseases include, but are not limited to, osteoporosis, rheumatoid arthritis, atherosclerosis, asthma (Ray & Cohn, (1999) J. Clin. Invest. 104, 985-993; Christman et al., (2000) Chest 117, 1482-1487) and Alzheimer's disease. For a review of diseases with an NF-κB inflammatory component, see Epstein, (1997) New Eng. J. Med. 336, 1066-1071; Lee et al., (1998) J. Clin. Pharmacol. 38, 981-993; Brand et al., (1997) Exp. Physiol. 82, 297-304.

Pathological processes associated with a pro-inflammatory response in which the anti-inflammatory compounds of the invention would be useful for treatment further include allergies such as allergic rhinitis, uticaria, anaphylaxis, drug sensitivity, food sensitivity and the like; cutaneous inflammation such as dermatitis, eczema, psoriasis, contact dermatitis, sunburn, aging, and the like; arthritis such as osteoarthritis, psoriatic arthritis, lupus, spondylarthritis and the like; chronic obstruction pulmonary disease and chronic inflammatory bowel disease. The anti-inflammatory compounds of the present invention may further be used to replace corticosteroids in any application in which corticosteroids are used including immunosuppression in transplants and cancer therapy.

As used herein, the term “subject” includes warm-blooded animals, preferably mammals, including humans. In a preferred embodiment, the subject is a primate. In an even more preferred embodiment, the primate is a human.

As used herein, the term “administering” to a subject includes dispensing, delivering or applying an anti-inflammatory compound, e.g., an anti-inflammatory compound in a pharmaceutical formulation (as described herein), to a subject by any suitable route for delivery of the compound to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route (e.g., by inhalation).

As used herein, the term “effective amount” includes an amount effective, at dosages and for periods of time necessary, to achieve the desired result, e.g., sufficient to treat an inflammatory disorder in a subject. An effective amount of an anti-inflammatory compound of the invention, as defined herein may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed by the therapeutically beneficial effects.

A therapeutically effective amount of an anti-inflammatory compound of the invention (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an anti-inflammatory compound of the invention can include a single treatment or, preferably, can include a series of treatments. In one example, a subject is treated with an anti-inflammatory compound of the invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of an anti-inflammatory compound of the invention used for treatment may increase or decrease over the course of a particular treatment.

The anti-inflammatory compounds of the present invention can be provided alone, or in combination with other agents that modulate a particular pathological process. For example, an anti-inflammatory compound of the present invention can be administered in combination with other known anti-inflammatory agents. Known anti-inflammatory agents that may be used in the methods of the invention can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell, N.J., Medical Economics Co., the complete contents of which are expressly incorporated herein by reference. The anti-inflammatory compounds of the invention and the additional anti-inflammatory agents may be administered to the subject in the same pharmaceutical composition or in different pharmaceutical compositions (at the same time or at different times). Suitable additional anti-inflammatory agents include, but are not limited to, anti-TNFα agents, such as etanercept and infliximab; cyclooxygenase-2 inhibitors, such as celecoxib and rofecoxib; steroidal agents, scu as dexamethasone and prednisone; non-steroidal anti-inflammatory agents, such as aspirin, acetominaphen, ibuprofen, naproxen, salicylic acid, and 5-aminosalicylate; and immune suppressants, such as cyclosporine and FK506.

Cell Membrane-Permeable Polybasic Peptides

The anti-inflammatory compounds used in the methods of the invention comprise polybasic peptides or analogues or derivatives thereof.

As used herein, the term “polybasic peptide”, e.g., cell membrane-permeable polybasic peptide, includes any of the polybasic peptides known in the art to facilitate transport of proteins and other molecules or moieties into cells. The polybasic peptide can comprise up to 40, 30, 25, 20, 15, 12, 10, 8 or 7 amino acid residues. For example, the peptide can include 5-40, 5-30, 5-25, 5-20, 5-15, 5-10, 7-20, 7-15, or 7-12 amino acid residues. Preferably, the polybasic peptide comprises 25 or fewer, 20 or fewer, 15 or fewer or 12 or fewer residues. Suitable peptides are known in the art and include the third helix of the antennapedia homeodomain protein and vaiants thereof, e.g., N-terminal truncated variants thereof; the HIV tat protein, particularly sequences including residues 48-57 of the tat protein; peptides derived from gelsolin; and synthetic peptides. Suitable peptides include those described in, for example, Derossi et al., (1994) J. Biol. Chem. 269, 10444-10450; Lindgren et al., (2000) Trends Pharmacol. Sci. 21, 99-103; Ho et al., Cancer Research 61, 474-477 (2001); U.S. Pat. No. 5,888,762; U.S. Pat. No. 6,015,787; U.S. Pat. No. 5,846,743; U.S. Pat. No. 5,747,641; U.S. Pat. No. 5,804,604, and published PCT applications WO 98/52614, WO 00/29427 and WO 99/29721, the contents of each of which are incorporated herein by reference in their entirety.

Suitable peptides include peptides having an amino acid sequence which includes multiple basic residues, and preferably, at least three, four, five, six, seven or more basic residues. The basic residues can be independently selected from arginine, lysine and non-natural amino acid residues having basic side chains. In one subset of peptides of the invention, at least 30%, 40%, 50%, 60%, 70%, 80% or 90% of the residues in the peptide are basic residues; in preferred embodiment, at least 30%, 40%, 50%, 60%, 70%, or 80% of the residues in the peptide are independently selected from lysine and arginine.

In one embodiment, the peptide used in the methods of the invention is of the formula B₁—X₁—X₂—X₃—B₂—X₄—X₅—B₃, where B₁, B₂ and B₃ are each, independently, a basic amino acid residue and X₁, X₂, X₃, X₄ and X₅ are each, independently, an alpha-helix promoting amino acid residue. In one embodiment, at least one of B₁, B₂ and B₃ is arginine; preferably B₁, B₂ and B₃ are each arginine. In another embodiment, at least one of X₁, X₂, X₃, X₄ and X₅ is alanine; preferably all of residues X₁-X₅ are alanine.

In another embodiment, the peptide used in the methods of the invention is of the formula B₁—X₁—X₂—B₂—B₃—X₃—X₄—B₄, where B₁, B₂, B₃ and B₄ are each, independently, a basic amino acid residue and X₁, X₂, X₃ and X₄ are each, independently, an alpha-helix promoting amino acid residue. In one embodiment, at least one of B₁, B₂, B₃ and B₄ is arginine; preferably B₁, B₂, B₃ and B₄ are each arginine. In another embodiment, at least one of X₁, X₂, X₃ and X₄ is alanine; preferably all of residues X₁-X₄ are alanine.

As used herein, an “alpha helix promoting amino acid residue” includes an amino acid residue which is known to form or stabilize an alpha helical structure. Preferred residues of this type include alanine, methionine, arginine, leucine and lysine. Preferably, the alpha helix promoting residue is alanine.

Specific examples of peptides which can be used in the methods of the invention include peptides having the sequences DRQIKIWFQNRRMKWKK (SEQ ID NO:1); RQIKIWFQNRRMKWKK (SEQ ID NO:2); QIKIWFQNRRMKWKK (SEQ ID NO:3); IKIWFQNRRMKWKK (SEQ ID NO:4); KIWFQNRRMKWKK (SEQ ID NO:5); IWFQNRRMKWKK (SEQ ID NO:6); WFQNRRMKWKK (SEQ ID NO:7); FQNRRMKWKK (SEQ ID NO:8); QNRRMKWKK (SEQ ID NO:9); NRRMKWKK (SEQ ID NO:10); RRMKWKK (SEQ ID NO:11); FKSGLKYKK (SEQ ID NO:12); KSGLKYKK (SEQ ID NO:13); QRLFQVKGRR (SEQ ID NO:14); RLFQVKGRR (SEQ ID NO:15); YGRKKRRQRRRP (SEQ ID NO:16); GRKKRRQRRRP (SEQ ID NO:17); RKKRRQRRRP (SEQ ID NO:18); RKKRRQRRRPGG (SEQ ID NO:19); AGRKKRRQARR (SEQ ID NO:20); YARKARRQARR (SEQ ID NO:21); YARAAARQARA (SEQ ID NO:22); YARAARRAARR (SEQ ID NO:23); YARAARRAARA (SEQ ID NO:24); YARRRRRRRRR (SEQ ID NO:25); RKKRRQRRR (SEQ ID NO:26); RKKRRQRR (SEQ ID NO:27); YGRKKRRQRRR (SEQ ID NO:28); YGRKKRRQRR (SEQ ID NO:29); GRKKRRQRRR (SEQ ID NO:30); GRKKRRQRR (SEQ ID NO:31); RRRRR (SEQ ID NO:32); RRRRRR (SEQ ID NO:33); RRRRRRR (SEQ ID NO:34); RRRRRRRR (SEQ ID NO:35); RRRRRRRRR (SEQ ID NO:36); RRRRRRRRRR (SEQ ID NO:37); RRRRRRRRRRR (SEQ ID NO:38); RRRRRRRRRRRR (SEQ ID NO:39).

The peptidic anti-inflammatory compounds used in the methods of the invention are preferably L-peptides, that is, each of the chiral amino acid residues within the peptide has an L-configuration. However, in one embodiment, the peptides also include one or more amino acid residues in the D-configuration. The peptides can also include other non-natural amino acid residues, including non-natural amino acid residues having basic or cationic side chains, for example, side chains which include primary, secondary, tertiary or quaternary amino groups, imino groups or guanidino groups.

The peptidic anti-inflammatory compounds used in the methods of the invention can optionally include modifying groups attached to the C-terminus, the N-terminus or both. For example, suitable modifying groups which can be attached to the C-terminus include substituted and unsubstituted amino groups, for example, —NH₂, —NH(alkyl) and —N(alkyl)₂ groups; and alkoxy groups, such as linear, branched or cyclic C₁-C₆-alkoxy groups. A preferred C-terminal modifying group is the —NH₂ group. Suitable modifying groups which can be attached to the N-terminus include acyl groups, such as the acetyl group; and alkyl groups, preferably C₁-C₆-alkyl groups, more preferably methyl. Further suitable modifying groups that may be attached to the anti-inflammatory compounds of the present invention include additional amino acid residues, e.g., up to 4, preferably 3, 2 or 1 amino acid residues. The additional amino acid residues may be attached to the C-terminus, the N-terminus or both.

As used herein, the terms “peptide compound” and “peptidic compound” are intended to include peptides comprised of naturally-occurring amino acids, as well as peptide derivatives, peptide analogues and peptide mimetics of the naturally-occurring amino acid structures. The terms “peptide analogue”, “peptide derivative” and “peptidomimetic” as used herein are intended to include molecules which mimic the chemical structure of a peptide and retain the functional properties of the peptide. Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

As used herein, a “derivative” of a compound X (e.g., a peptide or amino acid) refers to a form of X in which one or more reaction groups on the compound have been derivatized with a modifying (derivative) group. Examples of peptide derivatives include peptides in which an amino acid side chain, the peptide backbone, or the amino- or carboxy-terminus has been derivatized (e.g., peptidic compounds with methylated amide linkages).

An “analogue” of a reference amino acid, as the term is used herein, is an α- or β-amino acid having a side chain which is (a) the same as the side chain of the reference amino acid (when the analogue is a β-amino acid residue, a peptoid, or the D-amino acid enantiomer of the reference acid); (b) is an isomer of the side chain of the reference amino acid; (c) is a homologue of the side chain of the reference amino acid; (d) results from replacement of a methylene group in the side chain of the reference amino acid with a heteroatom or group selected from NH, O and S; (e) results from a simple substitution on the side chain of the reference amino acid or any of the preceding (a) to (c); and/or (f) results from a conservative substitution (discussed infra). Analogues of a reference amino acid further include the reference amino acid or any of (a)-(e) above in which the α-nitrogen atom is substituted by a lower alkyl group, preferably a methyl group. A “homologue” of the given amino acid is an α- or β-amino acid having a side chain which differs from the side chain of the given amino acid by the addition or deletion of from 1 to 4 methylene groups. A “simple substitution” of an amino acid side chain results from the substitution of a hydrogen atom in the side chain of the given amino acid with a small substituent, such as a lower alkyl group, preferably a methyl group; a halogen atom, preferably a fluorine, chlorine, bromine or iodine atom; or hydroxy.

Peptide mimetics that are structurally similar to therapeutically useful peptides may be used to produce an equivalent therapeutic or prophylactic effect. The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Generally, peptidomimetics are structurally similar to a paradigm peptide (i.e., a peptide that has a biological or pharmacological activity), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S. (1980) Trends Pharm. Sci. pp. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982) J. Chem. Soc. Perkin Trans. I. 307-314 (—CH—CH—, cis and trans); Almquist, R. G. et al. (1980) J. Med. Chem. 23:1392-1398 (—COCH2-); Jennings-White, C. et al. (1982) Tetrahedron Lett. 23:2533 (—COCH2-); Szelke, M. et al. European Appln. EP 45665 (1982) CA: 97:39405 (1982) (—CH(OH)CH2-); Holladay, M. W. et al. (1983) Tetrahedron Lett. 24:4401-4404 (—C(OH)CH2-); and Hruby, V. J. (1982) Life Sci. 31:189-199 (—CH2-S—); each of which is incorporated herein by reference. A particularly preferred non-peptide linkage is —CH2NH—.

Other examples of isosteres include peptides substituted with one or more benzodiazepine molecules (see, e.g., James, G. L. et al. (1993) Science 260:1937-1942). Other possible modifications include an N-alkyl (or aryl) substitution (ψ{CONR}), backbone crosslinking to construct lactams and other cyclic structures, substitution of all D-amino acids for all L-amino acids within the compound (“inverso” compounds) or retro-inverso amino acid incorporation (ψ{NHCO}). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al. “Perspectives in Peptide Chemistry” pp. 283-294 (1981). See also U.S. Pat. No. 4,522,752 by Sisto for further description of “retro-inverso” peptides. Other derivatives include C-terminal hydroxymethyl derivatives, O-modified derivatives (e.g., C-terminal hydroxymethyl benzyl ether) and N-terminally modified derivatives including substituted amides such as alkylamides and hydrazides.

Such peptide mimetics may have significant advantages over peptide embodiments, including, for example: more economical production, greater chemical stability, enhanced pharmacological properties (e.g., half-life, absorption, potency, efficacy, and the like), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivitization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) may be used to generate more stable peptides. In addition, constrained peptides may be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The term “conservative substitution”, as used herein, includes the replacement of one amino acid residue by another residue having similar side chain properties. As is known in the art, the twenty naturally amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group.

Pharmaceutical Preparations

The invention also includes pharmaceutical compositions comprising the anti-inflammatory compounds of the invention together with a pharmaceutically acceptable carrier. Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Gennaro et al., (1995) Remington's Pharmaceutical Sciences, Mack Publishing Company. In addition to the pharmacologically active agent, the compositions of the present invention may contain suitable pharmaceutically acceptable carriers comprising excipients and auxiliaries which facilitate processing of the active compounds into preparations which can be used pharmaceutically for delivery to the site of action. Suitable formulations for parenteral administration include aqueous solutions of the active compounds in water-soluble form, for example, water-soluble salts. In addition, suspensions of the active compounds as appropriate oily injection suspensions may be administered. Suitable lipophilic solvents or vehicles include fatty oils, for example, sesame oil or synthetic fatty acid esters, for example, ethyl oleate or triglycerides. Aqueous injection suspensions may contain substances which increase the viscosity of the suspension include, for example, sodium carboxymethyl cellulose, sorbitol, and dextran. Optionally, the suspension may also contain stabilizers. Liposomes can also be used to encapsulate the agent for delivery into the cell.

The pharmaceutical formulation for systemic administration according to the invention may be formulated for enteral, parenteral or topical administration. Indeed, all three types of formulations may be used simultaneously to achieve systemic administration of the active ingredient.

Suitable formulations for oral administration include hard or soft gelatin capsules, pills, tablets, including coated tablets, elixirs, suspensions, syrups or inhalations and controlled release forms thereof.

The anti-inflammatory compounds of the invention can also be incorporated into pharmaceutical compositions which allow for the sustained delivery of the anti-inflammatory compounds to a subject for a period of at least several weeks to a month or more. For example, the compounds of the invention can formulated as substantially insoluble ionic complexes of one or more biocompatible anionic carrier molecules, preferably, an anionic polymer. Such formulations are described in U.S. Pat. Nos. 5,968,895 and 6,180,608 B1, the contents of each of which are incorporated herein by reference in their entirety.

The anti-inflammatory compounds of the present invention may be administered via parenteral, subcutaneous, intravenous, intramuscular, intraperitoneal, transdermal or buccal routes. Alternatively, or concurrently, administration may be by the oral route or by inhalation or lavage, directly to the lungs. The dosage administered will be dependent upon the age, health, and weight of the recipient, kind of concurrent treatment, if any, frequency of treatment, and the nature of the effect desired.

The anti-inflammatory compounds used in the methods of treatment described herein may be administered systemically or topically, depending on such considerations as the condition to be treated, need for site-specific treatment, quantity of drug to be administered and similar considerations.

Topical administration may be used. Any common topical formation such as a solution, suspension, gel, ointment or salve and the like may be employed. Preparation of such topical formulations are well described in the art of pharmaceutical formulations as exemplified, for example, by Remington's Pharmaceutical Sciences. For topical application, these compounds could also be administered as a powder or spray, particularly in aerosol form. The active ingredient may be administered in pharmaceutical compositions adapted for systemic administration. As is known, if a drug is to be administered systemically, it may be confected as a powder, pill, tablet or the like or as a syrup or elixir for oral administration. For intravenous, intraperitoneal or intra-lesional administration, the compound will be prepared as a solution or suspension capable of being administered by injection. In certain cases, it may be useful to formulate these compounds in suppository form or as an extended release formulation for deposit under the skin or intramuscular injection. In a preferred embodiment, the anti-inflammatory compounds of the invention may be administered by inhalation. For inhalation therapy the compound may be in a solution useful for administration by metered dose inhalers or in a form suitable for a dry powder inhaler.

An effective amount is that amount which will modulate the activity or alter the level of a target protein. A given effective amount will vary from condition to condition and in certain instances may vary with the severity of the condition being treated and the patient's susceptibility to treatment. Accordingly, a given effective amount will be best determined at the time and place through routine experimentation. However, it is anticipated that in the treatment of a tumor in accordance with the present invention, a formulation containing between 0.001 and 5 percent by weight, preferably about 0.01 to 1 percent, will usually constitute a therapeutically effective amount. When administered systemically, an amount between 0.01 and 100 mg per kg body weight per day, but preferably about 0.1 to 10 mg per kg, will effect a therapeutic result in most instances.

In practicing the methods of this invention, the compounds of this invention may be used alone or in combination, or in combination with other therapeutic or diagnostic agents. In certain preferred embodiments, the compounds of this invention may be co-administered along with other compounds typically prescribed for these conditions according to generally accepted medical practice. The compounds of this invention can be utilized in vivo, ordinarily in mammals, preferably in humans.

In still another embodiment, the anti-inflammatory compounds of the invention may be coupled to chemical moieties, including proteins that alter the functions or regulation of target proteins for therapeutic benefit. These proteins may include in combination other inhibitors of cytokines and growth factors that may offer additional therapeutic benefit in the treatment of inflammatory disorders. In addition, the anti-inflammatory compounds of the invention may also be conjugated through phosphorylation to biotinylate, thioate, acetylate, iodinate using any of the cross-linking reagents well known in the art.

Screening Assays

In addition, this invention also provides screening methods for identifying anti-inflammatory compounds that may be used in the methods of the invention.

The peptidic anti-inflammatory compounds used in the methods of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

The peptidic anti-inflammatory compounds may then be evaluated for their anti-inflammatory activity using, for example, the lethal lipopolysaccharide mouse model or the Concanavalin A-induced hepatitis model described herein.

This invention is further illustrated by the following examples which should not be construed as limiting. The contents of all references, patents and published patent applications cited throughout this application, as well as the Figures and the Sequence Listing, are hereby incorporated by reference.

### EXAMPLES

### Example 1

### Synthesis of Anti-Inflammatory Peptides

Peptides were synthesized using known solid phase synthesis methods employing FMOC protection. Crude peptides were purified by liquid chromatography and characterized by mass spectrometry.

### Example 2

### Evaluation of Anti-Inflammatory Peptides In Lethal Lipopolysaccharide Mouse Model

In this experiment, the ability of the anti-inflammatory peptides of the invention to rescue mice challenged with a lethal amount of lipopolysaccharide (LPS) was assessed. LPS is a bacterial cell wall product that induces many of the responses that are seen in septic patients, including death. In this model, Salmonella typhimurium LPS in phosphate-buffered saline (PBS) was administered to male C57BL/6 mice by intravenous injection at a dose of 30 mg/kg (600 μg/20 g mouse). This dose was established in control experiments to be lethal in 100% of the mice that received it. Mice were treated with the test peptide by intravenous injection (in PBS) immediately prior to the LPS injection and 24 hours after the LPS injection. Mice were monitored twice daily for up to 8 days after receiving LPS and the duration of survival and the number of surviving mice were recorded.

The results of this study are presented in the following table which shows, for each dosing group, the number of mice surviving after 8 days.

| compound | survival (day 8) |
|---|---|
| vehicle | 0 |
| H-YARAARRAARR-NH₂ | 8 |
| H-yaraarraarr-NH₂ | 0 |
| H-RRAARRAARAY-NH₂ | 0 |
| H-rraarraaray-NH₂ | 0 |

The results presented in the Table 1 demonstrate that the L-peptide H—YARAARRAARR—NH₂ provides significant protection against lethal challenge with LPS in this model when administered at a dose of 5 mg/kg i.v. Neither the corresponding D-peptide, the corresponding retro peptide, H—RRAARRAARAY—NH₂, nor the corresponding retro-inverso peptide, H-rraarraaray-NH₂, showed any protective effect.

### Example 3

### Assessment of Anti-Inflammatory Peptides In Concanavalin A-Induced Hepatitis

In this experiment, the ability of anti-inflammatory peptides of the invention to rescue mice with Concanavalin A-induced hepatitis was determined.

Concanavalin A is a lectin, a class of proteins that bind to carbohydrates. When carbohydrates are part of a protein, the lectin binds to the protein. By binding to proteins on the cell surface, concanavalin A stimulates many cells, including T lymphocytes. In concert with other mediators that are released by concanavalin A stimulation, these T lymphocytes attack liver cells that also have concanavalin A bound to them, causing the liver cells to die. The involvement of T lymphocytes makes this model similar to human viral hepatitis. However, as part of this acute model, there is also a TNFα response.

The mice were placed in a restrainer and injected intravenously (i.v.) in the tail vein with test peptide or vehicle in PBS. The mice were then immediately injected i.v. with 15 mg/kg of concanavalin A dissolved in sterile PBS. The injection volume was 5 ml/kg (100 μl/20 g mouse) with a concanavalin A concentration of 3.0 mg/ml. The next morning (18-24 hours later), these mice were euthanized by CO₂ inhalation and blood was collected by cardiac puncture. The serum was then separated and analyzed for AST and ALT.

Study 1

Sixty-four male C57BL/6 mice weighing between 18 g and 22 g were divided into eight treatment groups of eight mice each as shown below.

| group | treatment |
|---|---|
| 1 | vehicle + vehicle iv |
| 2 | vehicle + vehicle sc |
| 3 | concanavalin A + vehicle iv |
| 4 | concanavalin A + vehicle sc |
| 5 | concanavalin A + 5 mg/kg H-YARAARRAARR-NH₂ iv |
| 6 | concanavalin A + 25 mg/kg H-YARAARRAARR-NH₂ sc |
| 7 | concanavalin A + 5 mg/kg H-yaraarraarr-NH₂ iv |
| 8 | concanavalin A + 25 mg/kg H-yaraarraarr-NH₂ sc |

The results of this study are shown in the table below. ALT and AST values are given as Sigma-Frankel units/ml, mean±SEM.

| Treatment | | |
|---|---|---|
| group | ALT | AST |
| 1 | 35 ± 5 | 94 ± 34 |
| 2 | 41 ± 7 | 173 ± 46 |
| 3 | 4000 ± 0 | 8000 ± 0 |
| 4 | 4000 ± 0 | 8000 ± 0 |
| 5 | 1789 ± 945 | 1982 ± 1032 |
| 6 | 4627 ± 677 | 7688 ± 268 |
| 7 | 5246 ± 876 | 7544 ± 717 |
| 8 | 4652 ± 697 | 7952 ± 373 |

The foregoing results show that H—YARAARRAARR—NH₂ is able to protect mice against concanavalin A-induced liver damage when administered intravenously at a dose of 5 mg/kg. This compound, however, had no protective effect when administered subcutaneously at a dose of 25 mg/kg. The D-peptide H-yaraarraarr-NH₂ was not protective at either dose or route of administration.

Study 2

Mice were divided into five treatment groups as indicated below.

| group | treatment |
|---|---|
| 1 | vehicle + vehicle iv |
| 2 | concanavalin A + vehicle iv |
| 3 | concanavalin A + H-RRMKWKK-NH₂ (5 mg/kg iv) |
| 4 | concanavalin A + H-rrmkwkk-NH₂ (5 mg/kg iv) |

The results of this study are shown in the table below. ALT and AST values are given as Sigma-Frankel units/ml, mean±SEM.

| Treatment | | |
|---|---|---|
| group | ALT | AST |
| 1 | 38 + 5 | 84 + 12 |
| 2 | 8656 + 4218 | |
| 3 | 1149 + 579 | 217 + 41 |
| 4 | 16421 + 1908 | |

The foregoing results show that H—RRMKWKK—NH₂ is able to protect against concanavalin A-induced liver damage when administered intravenously at a dose of 5 mg/kg. The corresponding D-peptide, H-rrmkwkk-NH₂ was not protective at this dose.

### Example 4

### Evaluation of Anti-Inflammatory Peptides For the Ability To Inhibit Lipopolysaccharide-Induced Secretion of Pro-Inflammatory Cytokines

In this experiment, the ability of anti-inflammatory peptides to inhibit lipopolysaccharide (LPS)-induced secretion of pro-inflammatory cytokines, specifically TNF-α, was assessed. In this model, male C57BL/6 mice were injected intravenously (i.v.) in the tail vein with test peptide or vehicle in PBS. The mice were then immediately injected i.v. with a sublethal dose of Salmonella typhimurium LPS (1 mg/kg, 20 μg/20 g mouse) in PBS. Mice were euthanized by CO₂ inhalation one hour post anti-inflammatory peptide and LPS injection (this time point was established in control experiments to yield peak TNF-α serum levels, and is consistent with data reported in the literature), blood was collected by cardiac puncture. The serum was then separated, and TNF-α was quantitated by ELISA.

Twenty-eight male C57BL/6 mice weighing between 18 g and 22 g were divided into 4 treatment groups with eight mice each, except group 1 with four mice, as shown below.

| group | treatment |
|---|---|
| 1 | vehicle + vehicle iv |
| 2 | vehicle + LPS (1 mg/kg) iv |
| 3 | LPS (1 mg/kg) + H-YARAARRAARR-NH₂ (5 mg/kg) iv |
| 4 | LPS (1 mg/kg) + H-RRMKWKK-NH₂ (5 mg/kg) iv |

The results of this study are shown in the table below. TNF-α serum levels are given as pg/ml (mean+−/SEM), relative TNF-α serum levels are given in percent (%), relative to the value of group 2 (100%).

| treatment | | Relative |
|---|---|---|
| group | TNF-α (serum) | TNF-α (serum) |
| 1 | <7.8* | 0 |
| 2 | 7308 +/− 576 | 100 |
| 3 | 3153 +/− 650 | 43 |
| 4 | 3012 +/− 375 | 41 |

The foregoing results demonstrate that H-YARAARRAARR-NH₂ and H-RRMKWKK-NH₂ are able to inhibit LPS-induced secretion of the proinflammatory cytokine, TNF-α when administered intravenously at a dose of 5 mg/kg.
*below limit of detection

Equivalents

Those skilled in the art will recognize, or be able to ascertain using no more than routine experimentation, many equivalents to the specific embodiments of the invention described herein. Such equivalents are intended to be encompassed by the following claims.

1. A method for treating an inflammatory disorder in a subject comprising administering to a subject a polybasic peptide comprising 25 or fewer amino acid residues in an amount effective to treat an inflammatory disorder.

2. The method of claim 1, wherein the polybasic peptide comprises from 5 to 16 amino acid residues.

3. The method of claim 1, wherein the polybasic peptide comprises from 5 to 12 amino acid residues.

4. The method of claim 1, wherein the polybasic peptide comprises from 7 to 11 amino acid residues.

5. The method of claim 1, wherein the polybasic peptide comprises the third helix of the antennapedia homeodomain protein or a fragment or variant thereof.

6. The method of claim 1, wherein the polybasic peptide comprises amino acid residues 48-57 of the HIV tat protein.

7. The method of claim 1, wherein the polybasic peptide is derived from gelsolin.

8. The method of claim 1, wherein at least 50% percent of the amino acid residues in the polybasic peptide are independently selected from lysine and arginine residues.

9. A method for treating an inflammatory disorder in a subject comprising administering to a subject a polybasic peptide having the structure:
B₁—X₁—X₂—X₃—B₂—X₄—X₅—B₃,
wherein B₁, B₂ and B₃ are each, independently, a basic amino acid residue and X₁, X₂, X₃, X₄ and X₅ are each, independently, an alpha-helix promoting amino acid residue, in an amount effective to treat an inflammatory disorder.

10. The method of claim 9, wherein at least one of B₁, B₂ and B₃ is an arginine residue.

11. The method of claim 9, wherein each of B₁, B₂ and B₃ is an arginine residue.

12. The method of claim 9, wherein at least one of X₁, X₂, X₃, X₄ and X₅ is an alanine residue.

13. The method of claim 9, wherein X₁, X₂, X₃, X₄ and X₅ are alanine residues.

14. The method of claim 9, wherein the polybasic peptide further comprises a modifying group.

15. The method of claim 14, wherein the modifying group is selected from the group consisting of an —NH₂ group; an —NH(alkyl) group; an —N(alkyl)₂ group; an alkoxy group; an acyl group; and an alkyl group.

16. A method for treating an inflammatory disorder in a subject comprising administering to a subject a polybasic peptide having the structure:
B₁—X₁—X₂—B₂—B₃—X₃—X₄—B₄,
wherein B₁, B₂, B₃ and B₄ are each, independently, a basic amino acid residue and X₁, X₂, X₃ and X₄ are each, independently, an alpha-helix promoting amino acid residue, in an amount effective to treat an inflammatory disorder.

17. The method of claim 16, wherein at least one of B₁, B₂, B₃ and B₄ is an arginine residue.

18. The method of claim 16, wherein each of B₁, B₂, B₃ and B₄ is an arginine residue.

19. The method of claim 16, wherein at least one of X₁, X₂, X₃ and X₄ is an alanine residue.

20. The method of claim 16, wherein each of X₁, X₂, X₃ and X₄ is an alanine residue.

21. The method of claim 16, wherein the polybasic peptide further comprises a modifying group.

22. The method of claim 21, wherein the modifying group is selected from the group consisting of an —NH₂ group; an —NH(alkyl) group; an —N(alkyl)₂ group; an alkoxy group; an acyl group; and an alkyl group.

23. A method for treating an inflammatory disorder in a subject comprising administering to a subject a polybasic peptide having a structure selected from the group consisting of: DRQIKIWFQNRRMKWKK (SEQ ID NO:1); RQIKIWFQNRRMKWKK (SEQ ID NO:2); QIKIWFQNRRMKWKK (SEQ ID NO:3); IKIWFQNRRMKWKK (SEQ ID NO:4); KIWFQNRRMKWKK (SEQ ID NO:5); IWFQNRRMKWKK (SEQ ID NO:6); WFQNRRMKWKK (SEQ ID NO:7); FQNRRMKWKK (SEQ ID NO:8); QNRRMKWKK (SEQ ID NO:9); NRRMKWKK (SEQ ID NO:10); RRMKWKK (SEQ ID NO:11); FKSGLKYKK (SEQ ID NO:12); KSGLKYKK (SEQ ID NO:13); QRLFQVKGRR (SEQ ID NO:14); RLFQVKGRR (SEQ ID NO:15); YGRKKRRQRRRP (SEQ ID NO:16); GRKKRRQRRRP (SEQ ID NO:17); RKKRRQRRRP (SEQ ID NO:18); RKKRRQRRRPGG (SEQ ID NO:19); AGRKKRRQARR (SEQ ID NO:20); YARKARRQARR (SEQ ID NO:21); YARAAARQARA (SEQ ID NO:22); YARAARRAARR (SEQ ID NO:23); YARAARRAARA (SEQ ID NO:24); YARRRRRRRRR (SEQ ID NO:25); RKKRRQRRR (SEQ ID NO:26); RKKRRQRR (SEQ ID NO:27); YGRKKRRQRRR (SEQ ID NO:28); YGRKKRRQRR (SEQ ID NO:29); GRKKRRQRRR (SEQ ID NO:30); GRKKRRQRR (SEQ ID NO:31); RRRRR (SEQ ID NO:32); RRRRRR (SEQ ID NO:33); RRRRRRR (SEQ ID NO:34); RRRRRRRR (SEQ ID NO:35); RRRRRRRRR (SEQ ID NO:36); RRRRRRRRRR (SEQ ID NO:37); RRRRRRRRRRR (SEQ ID NO:38); and RRRRRRRRRRRR (SEQ ID NO:39), in an amount effective to treat an inflammatory disorder.

24. An anti-inflammatory compound having the structure:
B₁—X₁—X₂—X₃—B₂—X₄—X₅—B₃,
wherein B₁, B₂ and B₃ are each, independently, a basic amino acid residue and X₁, X₂, X₃, X₄ and X₅ are each, independently, an alpha-helix promoting amino acid residue.

25. The anti-inflammatory compound of claim 24, wherein at least one of B₁, B₂ and B₃ is an arginine residue.

26. The anti-inflammatory compound of claim 24, wherein each of B₁, B₂ and B₃ is an arginine residue.

27. The anti-inflammatory compound of claim 24, wherein at least one of X₁, X₂, X₃, X₄ and X₅ is an alanine residue.

28. The anti-inflammatory compound of claim 24, wherein X₁, X₂, X₃, X₄ and X₅ are alanine residues.

29. An anti-inflammatory compound having the structure:
B₁—X₁—X₂—B₂—B₃—X₃—X₄—B₄,
wherein B₁, B₂, B₃ and B₄ are each, independently, a basic amino acid residue and X₁, X₂, X₃ and X₄ are each, independently, an alpha-helix promoting amino acid residue.

30. The anti-inflammatory compound of claim 29, wherein at least one of B₁, B₂, B₃ and B₄ is an arginine residue.

31. The anti-inflammatory compound of claim 29, wherein each of B₁, B₂, B₃ and B₄ is an arginine residue.

32. The anti-inflammatory compound of claim 29, wherein at least one of X₁, X₂, X₃ and X₄ is an alanine residue.

33. The anti-inflammatory compound of claim 29, wherein each of X₁, X₂, X₃ and X₄ is an alanine residue.

34. An anti-inflammatory compound having a structure selected from the group consisting of: DRQIKIWFQNRRMKWKK (SEQ ID NO:1); RQIKIWFQNRRMKWKK (SEQ ID NO:2); QIKIWFQNRRMKWKK (SEQ ID NO:3); IKIWFQNRRMKWKK (SEQ ID NO:4); KIWFQNRRMKWKK (SEQ ID NO:5); IWFQNRRMKWKK (SEQ ID NO:6); WFQNRRMKWKK (SEQ ID NO:7); FQNRRMKWKK (SEQ ID NO:8); QNRRMKWKK (SEQ ID NO:9); NRRMKWKK (SEQ ID NO:10); RRMKWKK (SEQ ID NO:11); FKSGLKYKK (SEQ ID NO:12); KSGLKYKK (SEQ ID NO:13); QRLFQVKGRR (SEQ ID NO:14); RLFQVKGRR (SEQ ID NO:15); YGRKKRRQRRRP (SEQ ID NO:16); GRKKRRQRRRP (SEQ ID NO:17); RKKRRQRRRP (SEQ ID NO:18); RKKRRQRRRPGG (SEQ ID NO:19); AGRKKRRQARR (SEQ ID NO:20); YARKARRQARR (SEQ ID NO:21); YARAAARQARA (SEQ ID NO:22); YARAARRAARR (SEQ ID NO:23); YARAARRAARA (SEQ ID NO:24); YARRRRRRRRR (SEQ ID NO:25); RKKRRQRRR (SEQ ID NO:26); RKKRRQRR (SEQ ID NO:27); YGRKKRRQRRR (SEQ ID NO:28); YGRKKRRQRR (SEQ ID NO:29); GRKKRRQRRR (SEQ ID NO:30); GRKKRRQRR (SEQ ID NO:31); RRRRR (SEQ ID NO:32); RRRRRR (SEQ ID NO:33); RRRRRRR (SEQ ID NO:34); RRRRRRRR (SEQ ID NO:35); RRRRRRRRR (SEQ ID NO:36); RRRRRRRRRR (SEQ ID NO:37); RRRRRRRRRRR (SEQ ID NO:38); and RRRRRRRRRRRR (SEQ ID NO:39).

35. A method for modulating the secretion of pro-inflammatory cytokines in a cell, the method comprising contacting a cell with a polybasic peptide in an amount effective to modulate the secretion of pro-inflammatory cytokines in a cell.

36. The method of claim 35, wherein said pro-inflammatory cytokine is TNF-α.

37. The method of claim 35, wherein the secretion of pro-inflammatory cytokines in a cell is inhibited.

38. The method of claim 35, wherein said polybasic peptide is an anti-inflammatory compound having a structure selected from the group consisting of: DRQIKIWFQNRRMKWKK (SEQ ID NO:1); RQIKIWFQNRRMKWKK (SEQ ID NO:2); QIKIWFQNRRMKWKK (SEQ ID NO:3); IKIWFQNRRMKWKK (SEQ ID NO:4); KIWFQNRRMKWKK (SEQ ID NO:5); IWFQNRRMKWKK (SEQ ID NO:6); WFQNRRMKWKK (SEQ ID NO:7); FQNRRMKWKK (SEQ ID NO:8); QNRRMKWKK (SEQ ID NO:9); NRRMKWKK (SEQ ID NO:10); RRMKWKK (SEQ ID NO:11); FKSGLKYKK (SEQ ID NO:12); KSGLKYKK (SEQ ID NO:13); QRLFQVKGRR (SEQ ID NO:14); RLFQVKGRR (SEQ ID NO:15); YGRKKRRQRRRP (SEQ ID NO:16); GRKKRRQRRRP (SEQ ID NO:17); RKKRRQRRRP (SEQ ID NO:18); RKKRRQRRRPGG (SEQ ID NO:19); AGRKKRRQARR (SEQ ID NO:20); YARKARRQARR (SEQ ID NO:21); YARAAARQARA (SEQ ID NO:22); YARAARRAARR (SEQ ID NO:23); YARAARRAARA (SEQ ID NO:24); YARRRRRRRRR (SEQ ID NO:25); RKKRRQRRR (SEQ ID NO:26); RKKRRQRR (SEQ ID NO:27); YGRKKRRQRRR (SEQ ID NO:28); YGRKKRRQRR (SEQ ID NO:29); GRKKRRQRRR (SEQ ID NO:30); GRKKRRQRR (SEQ ID NO:31); RRRRR (SEQ ID NO:32); RRRRRR (SEQ ID NO:33); RRRRRRR (SEQ ID NO:34); RRRRRRRR (SEQ ID NO:35); RRRRRRRRR (SEQ ID NO:36); RRRRRRRRRR (SEQ ID NO:37); RRRRRRRRRRR (SEQ ID NO:38); and RRRRRRRRRRRR (SEQ ID NO:39).


SEQUENCE LISTING (39 sequences)

SEQ ID NO: 1 - protein, 17 aa, Artificial Sequence
  anti-inflammatory compound
DRQIKIWFQNRRMKWKK

SEQ ID NO: 2 - protein, 16 aa, Artificial Sequence
  anti-inflammatory compound
RQIKIWFQNRRMKWKK

SEQ ID NO: 3 - protein, 15 aa, Artificial Sequence
  anti-inflammatory compound
QIKIWFQNRRMKWKK

SEQ ID NO: 4 - protein, 14 aa, Artificial Sequence
  anti-inflammatory compound
IKIWFQNRRMKWKK

SEQ ID NO: 5 - protein, 13 aa, Artificial Sequence
  anti-inflammatory compound
KIWFQNRRMKWKK

SEQ ID NO: 6 - protein, 12 aa, Artificial Sequence
  anti-inflammatory compound
IWFQNRRMKWKK

SEQ ID NO: 7 - protein, 11 aa, Artificial Sequence
  anti-inflammatory compound
WFQNRRMKWKK

SEQ ID NO: 8 - protein, 10 aa, Artificial Sequence
  anti-inflammatory compound
FQNRRMKWKK

SEQ ID NO: 9 - protein, 9 aa, Artificial Sequence
  anti-inflammatory compound
QNRRMKWKK

SEQ ID NO: 10 - protein, 8 aa, Artificial Sequence
  anti-inflammatory compound
NRRMKWKK

SEQ ID NO: 11 - protein, 7 aa, Artificial Sequence
  anti-inflammatory compound
RRMKWKK

SEQ ID NO: 12 - protein, 9 aa, Artificial Sequence
  anti-inflammatory compound
FKSGLKYKK

SEQ ID NO: 13 - protein, 8 aa, Artificial Sequence
  anti-inflammatory compound
KSGLKYKK

SEQ ID NO: 14 - protein, 10 aa, Artificial Sequence
  anti-inflammatory compound
QRLFQVKGRR

SEQ ID NO: 15 - protein, 9 aa, Artificial Sequence
  anti-inflammatory compound
RLFQVKGRR

SEQ ID NO: 16 - protein, 12 aa, Artificial Sequence
  anti-inflammatory compound
YGRKKRRQRRRP

SEQ ID NO: 17 - protein, 11 aa, Artificial Sequence
  anti-inflammatory compound
GRKKRRQRRRP

SEQ ID NO: 18 - protein, 10 aa, Artificial Sequence
  anti-inflammatory compound
RKKRRQRRRP

SEQ ID NO: 19 - protein, 12 aa, Artificial Sequence
  anti-inflammatory compound
RKKRRQRRRPGG

SEQ ID NO: 20 - protein, 11 aa, Artificial Sequence
  anti-inflammatory compound
AGRKKRRQARR

SEQ ID NO: 21 - protein, 11 aa, Artificial Sequence
  anti-inflammatory compound
YARKARRQARR

SEQ ID NO: 22 - protein, 11 aa, Artificial Sequence
  anti-inflammatory compound
YARAAARQARA

SEQ ID NO: 23 - protein, 11 aa, Artificial Sequence
  anti-inflammatory compound
YARAARRAARR

SEQ ID NO: 24 - protein, 11 aa, Artificial Sequence
  anti-inflammatory compound
YARAARRAARA

SEQ ID NO: 25 - protein, 11 aa, Artificial Sequence
  anti-inflammatory compound
YARRRRRRRRR

SEQ ID NO: 26 - protein, 9 aa, Artificial Sequence
  anti-inflammatory compound
RKKRRQRRR

SEQ ID NO: 27 - protein, 8 aa, Artificial Sequence
  anti-inflammatory compound
RKKRRQRR

SEQ ID NO: 28 - protein, 11 aa, Artificial Sequence
  anti-inflammatory compound
YGRKKRRQRRR

SEQ ID NO: 29 - protein, 10 aa, Artificial Sequence
  anti-inflammatory compound
YGRKKRRQRR

SEQ ID NO: 30 - protein, 10 aa, Artificial Sequence
  anti-inflammatory compound
GRKKRRQRRR

SEQ ID NO: 31 - protein, 9 aa, Artificial Sequence
  anti-inflammatory compound
GRKKRRQRR

SEQ ID NO: 32 - protein, 5 aa, Artificial Sequence
  anti-inflammatory compound
RRRRR

SEQ ID NO: 33 - protein, 6 aa, Artificial Sequence
  anti-inflammatory compound
RRRRRR

SEQ ID NO: 34 - protein, 7 aa, Artificial Sequence
  anti-inflammatory compound
RRRRRRR

SEQ ID NO: 35 - protein, 8 aa, Artificial Sequence
  anti-inflammatory compound
RRRRRRRR

SEQ ID NO: 36 - protein, 9 aa, Artificial Sequence
  anti-inflammatory compound
RRRRRRRRR

SEQ ID NO: 37 - protein, 10 aa, Artificial Sequence
  anti-inflammatory compound
RRRRRRRRRR

SEQ ID NO: 38 - protein, 11 aa, Artificial Sequence
  anti-inflammatory compound
RRRRRRRRRRR

SEQ ID NO: 39 - protein, 12 aa, Artificial Sequence
  anti-inflammatory compound
RRRRRRRRRRRR